(19) United States
(12) Patent Application Publication
(10) Pub. No.: US 2016/0327567 A1
(43) Pub. Date: Nov. 10, 2016

(54) METHODS AND DEVICES FOR ALLERGY TESTING AND TREATMENT

(71) Applicant: Spiriplex, Inc, Lincolnshire, IL (US)

(72) Inventor: David C. Fleisner, Buffalo Grove, IL (US)

(21) Appl. No.: 14/706,590

(22) Filed: May 7, 2015

(51) Int. Cl. G01N 33/68

(52) U.S. Cl. CPC G01N 33/6854; G01N 2800/24; G01N 2800/60

(57) ABSTRACT

A method of testing a patient for allergies includes obtaining from the patient a blood sample having an original volume of at most 0.5 ml, and testing the blood sample for IgE reactive with antigens from a plurality of allergen sources by multiple ELISA. The blood sample may be taken from the patient in a clinic, and/or the blood sample may be taken by a physician without venipuncture. The blood sample may be dried. A protein array for testing blood for IgE may be used in the multiple ELISA, and includes a plurality of regions or spots on a surface, each region or spot containing a mixture of different proteins from a single allergen source. If the patient is determined to have allergies, the patient may be treated by allergy immunotherapy, including administering to the patient the at least one allergen source.

### BACKGROUND

Allergic reactions occur when a person's (human's) immune system reacts to normally harmless substances in the environment. Allergic reactions include activation of mast cells and basophils by immunoglobulin E antibodies (IgE). The reaction results in an inflammatory response which can sometime be dangerous, such as in the case of anaphylactic reaction, but is almost always uncomfortable to the patient with symptoms such as urticaria (hives), allergic conjunctivitis (red itchy eyes), and rhinorrhea (runny nose). An allergic reaction can also lead to an asthma attack (asthma exacerbation) in patients with asthma.

Allergy diagnosis has been available for many years. There are two general types of allergy testing: skin tests and blood tests. Skin tests, such as puncture testing and patch testing, involve applying different allergen sources to different locations on or in the skin. If the patient is allergic to the allergen source applied, an inflammatory reaction will occur at that location on the skin of the patient. Proper interpretation of skin tests requires a physician who is an allergy specialist (an allergist). Furthermore, skin tests can occasionally result in a serious life-threatening anaphylactic reaction, so an allergist should only carry out skin tests in a facility prepared to treat such a reaction, for example in or adjacent to a hospital. In addition, if the patient has used (or has been administered) anti-inflammatory medication, such as anti-histamines or steroids, a skin test will not be accurate until about 3 weeks after such administration.

Blood tests may also be used for allergy diagnosis. A blood test is carried out by a medical testing laboratory, and examines blood samples of a patient (typically 3 to 4 vials of blood) for the concentration of IgE which are reactive with specific proteins found in different allergen sources: the higher the concentration of each IgE antibody, the greater the likelihood of symptoms from the proteins with which that antibody reacts (and its associated allergen source). Various IgE antibodies found at low levels may not be indicative of symptoms which would result from exposure of the patient to the corresponding allergen source, but can nevertheless help predict future allergy development.

Common types of tests for specific antibodies in blood are enzyme-linked immunosorbent assays (ELISA), and modified ELISA (also referred to simply as “ELISA” even though they may not employ an enzyme-substrate reaction for detection of an analyte). For example, in order to detect an antibody specific for an antigen (usually a protein) in a patient's blood, the antigen is immobilized on a surface, and then the surface is exposed to serum from the patient containing antibodies. Antibodies specific for the antigen bind to the antigen, and are therefore also immobilized on the surface. After washing away other antibodies which do not bind to the antigen, the surface is exposed to secondary antibodies linked to a reporter molecule, which bind to the bound antibody. The reporter molecule is then activated, to identify the bound antibody and preferably quantify the amount of bound antibody. In traditional ELISA, the reporter molecule is an enzyme which is allowed to react with a substrate for the enzyme. In modified ELISA, the reporter molecule may be, for example, a fluorescent dye. ELISA has been used, for example, to detect HIV antibodies in blood. In this case, HIV antigens are bound to a plate, the patient's serum is exposed to the plate, and a secondary antibody linked to a reporter molecule is allowed to bind to any antibody which has bound to the plate. The reporter molecule is then activated, in order to quantify the amount of the patient's antibodies which reacted with the HIV antigens bound to the plate.

Multiple ELISA may be used to detect or quantify simultaneously more than one antibody in a patient's blood sample. In multiple ELISA, different antigens are bound at different location on the surface. If fluorescent reporter molecules are used, then the amount of antibody bound to each location may be determined by examination of the surface with a fluorometer. For example, a 96-well plate or microarray may be used as a surface for multiple ELISA, with each well of the plate, or spot of the microarray, containing a different antigen. In a variation, a collection of beads may be used as the surface, with a different antigen bound to each bead.

In order to ensure reproducibility and specificity of ELISA for detecting and quantifying antibodies, a pure antigen is used so only the antibody of interest will become bound, and the amount of antigen used is in excess of the amount needed to bind all the antibody of interest which could be present in the patient's blood sample exposed to the surface. In multiple ELISA, it is preferable to use the same amount of each antigen in each location, to improve reproducibility. In the case of testing blood for IgE, each allergen source (such as peanut, dust mite, cat dander and golden rod pollen) contains many different proteins, with each protein being a different possible antigen. Due to the difficulty of isolating individual proteins in sufficient quantity from these natural sources, recombinantly produced proteins are mostly used as the antigens, and at most only one or a small number of proteins from each allergen source are used. An example of such a multiple ELISA for IgE analysis is IMMUNOCAP® Specific IgE Blood Test (available from Thermo Fisher Scientific (Milwaukee, Wis.) and produced by Phadia AB (Uppsala, Sweden), with testing through QUEST DIAGNOSTICS® (Madison, N.J.)). At least 2 milliliters (ml) of serum from a patient are necessary for IgE testing using the IMMUNOCAP® Specific IgE Blood Test for each set of allergen sources.

The blood samples collected from patients (for allergy testing, as well as for other diagnostic tests) must be handled as biohazard material, must be kept refrigerated and must be tested quickly, or the test results will be inaccurate due to chemical reactions which degrade IgE or interfere with ELISA. In order to ensure proper handling of blood samples and timely delivery to testing centers, a specialized and dedicated logistics service has been developed by companies such as QUEST DIAGNOSTICS®, which quickly collects blood sample and delivers them to testing center, keeping the samples refrigerated and properly secured to minimize any biohazard. The logistics service must exist in every location where blood samples may be collected from patients. Furthermore, there must be a testing center close enough to where the blood sample was collected so that the blood sample may be analyzed no later than the day after the sample was collected (next-day analysis of blood samples is considered the standard to ensure accuracy). Although this logistics service and network of testing centers is used for blood samples for most blood tests (not just for allergy testing), it adds an enormous cost because of the high cost of transporting blood samples and the large investment in equipment, properly training personnel, and the need for many small testing centers rather than national or regional testing centers. Furthermore, it is not cost effective to have the logistics service and network of testing centers in many rural or isolated locations, so patients located in such places must travel to a laboratory for a blood draw which is in a location served by the logistics service and associated testing centers, further eroding patient compliance.

Prior to diagnosis with allergies, most patients first visit a primary care physician. When a primary care physician suspects that a patient may have allergies, he or she will then want to test the patient for allergies. In most cases, the primary care physician will refer the patent to an allergist, who will then either carry out a skin test or a blood test for allergies. Some primary care physicians will order a blood test for allergies without referral to an allergist, however a skin test for allergies requires an allergist for proper interpretation and carries the risk of a serious life-threatening anaphylactic reaction and cannot be carried out by a primary care physician. Many primary care physicians and allergists operate out of clinics, and will direct patients to a laboratory in a different location in order to have the blood drawn (typically 3 to 4 vials) necessary for a blood test for allergies, by venipuncture. Drawing blood by venipuncture is carried out by a phlebotomist, a person specifically trained for this activity; some states, such as California and Washington require a special state certificate in order to be a phlebotomist.

Significant costs are associated with proper allergy testing. Furthermore, at each stage of referral or testing, additional costs are incurred and the likelihood of completing the testing process is reduced due to patient non-compliance. For example, a patient is typically first seen by a primary care physician, who will then refer the patient to an allergist. The patient must then make an appointment and visit the allergist, typically paying a co-payment for the visit, and incurring additional insurance costs. If the allergist orders a blood test for allergies, the patient will then need to visit a laboratory for a blood draw by venipuncture, incurring further patient costs as well additional insurance costs. Even if the primary care physician directly orders a blood test for allergies, the patient will likely still need to visit a laboratory for a blood draw by venipuncture—a survey has indicated that when patients must visit a laboratory for a blood draw after a blood test is ordered by a primary care physician, about 40-50% do not follow through with the laboratory visit. Each stage increases costs, and requires patient compliance to make appointments with an allergist and/or visit a laboratory. Co-payments required of patients are typically greater for specialists, and may act as a disincentive for patient compliance. May allergies are seasonal, so unless the patient acts quickly he or she may have temporary symptom relief, further eroding patient compliance. Yet the costs associated with undiagnosed allergies can be even greater: additional physician consultations, referrals to secondary care, misdiagnosis, allergy symptoms causing patient suffering and missed work, and emergency admissions due to life-threatening anaphylactic reaction or asthma attack. Improvements in allergy testing which result in improved patient compliance and lower costs are therefore needed.

There are 3 main techniques to treat allergies: avoidance of allergen sources, medications to treat allergy symptoms, and allergy immunotherapy. Avoidance is available in some instances (for example, cat or dog allergies), but not in others (such as seasonal allergies to plant or tree pollens). Medication to treat allergy symptoms is effective in some patient, and a variety of medications are available, but in many cases the effectiveness of the medication may lessen over time, and some patients cannot tolerate the side effects of the medications. Allergy immunotherapy, unlike avoidance and medications to treat allergy symptoms, offers the possibility of curing the patient of allergies, or at least dramatically reducing the symptoms.

Allergy immunotherapy includes exposing the patient to small amounts of the allergen source over a period time, resulting in desensitization of the patient to the allergen source. Traditionally, administration has been by injection into the skin, starting with a small amount of the allergen which is then increased over time. Sublingual administration is also possible. Administration continues during the maintenance phase of the therapy. Allergy immunotherapy has also been shown to reduce the risk of developing asthma in some patients, and prevent the development of new sensitivities in people who already have allergies. Allergen sources are typically administered as extracts of the allergen sources, which contain a complex mixture of proteins and other molecules extracted from the allergen source. Extracts of allergen sources are commercially available (for example, from GREER® Allergy Immunotherapy (Lenoir, N.C.), Human Allergy Products and Services). The most preferred type of allergy immunotherapy starts with a skin test using standardized extracts of allergen sources, followed by administration of the same standardized extracts of the allergen sources to which the patient reacted; this matching between the standardized extracts used in the skin test and the allergy immunotherapy is believed to provide the best patient outcomes.

### SUMMARY

In a first aspect, the present invention is a method of testing a patient for allergies, comprising obtaining from the patient a blood sample having an original volume of at most 0.5 ml, and testing the blood sample for IgE reactive with antigens from a plurality of allergen sources by multiple ELISA. The blood sample was taken from the patient in a clinic, and/or the blood sample was taken by a physician without venipuncture.

In a second aspect, the present invention is a protein array for testing blood for IgE, comprising a plurality of regions or spots on a surface, each region or spot comprising a mixture of different proteins from a single allergen source.

In a third aspect, the present invention is a method of treating a patient having allergies with allergy immunotherapy, comprising testing the patient for allergies by the method noted above, to identify at least one allergen source to which the patient is allergic, and administering to the patient the at least one allergen source.

In a fourth aspect, the present invention is a method of treating a patient having allergies with allergy immunotherapy, comprising testing a blood sample from the patient for IgE reactive with antigens from a plurality of allergen sources by multiple ELISA, to identify at least one allergen source to which the patient is allergic, and administering to the patient the at least one allergen source. The multiple ELISA is carried out with a protein array comprising a plurality of regions or spots on a surface, each region or spot comprising a mixture of different proteins from a single allergen source.

In a fifth aspect, the present invention is a method of testing a patient for allergies, comprising obtaining from the patient a blood sample having an original volume of at most 0.5 ml, drying the blood sample, and testing the blood sample for IgE reactive with antigens from a plurality of allergen sources by multiple ELISA.

### DEFINITIONS

An allergen source is an organism (such as dust mites) or part of an organism (such as cat dander), or a product produced from an organism (such as latex), which contains a plurality of proteins (at least 2, and preferably at least 3, at least 5 or at least 10, or more) and optionally other compounds. An allergen source causes allergies in at least one person, and is not an isolated and purified protein, and is not produced recombinantly. Examples of allergen sources include insects, mammals, plants and plant pollens, as well as foods.

An allergen source extract (or extract of an allergen source) is prepared from the corresponding allergen source, and contains a plurality of proteins (at least 2, and preferably at least 3, at least 5 or at least 10, or more) and optionally other compounds, which originate from the allergen source. Allergen source extracts may optionally contain other added ingredient, for example solvents (such as water, alcohols, glycerin, and oil) and/or preservatives. Preferably, the allergen source extract is a standardized allergen source extract (or standardized extract of the allergen source).

A primary care physician, as used herein, is a physician who in not trained or qualified to carry out a skin test for allergies, and is not an allergist, nor another type of specialist.

An allergist, as used herein, is a physician who is trained or qualified to carry out a skin test for allergies.

An antigen is a single chemical compound to which an antibody will bind, specifically and selectively. An epitope is that part of an antigen to which an antibody specifically and selectively binds. Occasionally, an antibody will be cross-reactive, and specifically and selectively bind to more than one antigen, because of the presence of an epitope common to both antigens.

Blood refers to whole blood or serum. Whole blood refers to blood which contains red blood cells and white blood cells, as well as antibodies and other proteins. Serum refers to blood from which cells have been removed, but still contains proteins such as antibodies. Blood, whole blood and serum may contain water, or may be dried.

A clinic, as used herein, is a facility where patients are treated and which does not employ a phlebotomist for obtaining blood by venipuncture. Examples of clinics include a physician office which does not employ a phlebotomist; a pharmacy wellness center which does not employ a phlebotomist; an chiropractic center which includes tables and rooms for treating patients and which does not employ a phlebotomist; a physical therapy practice which includes rooms and equipment for treating patients and which does not employ a phlebotomist; a nursing home which does not employ a phlebotomist; and a rehabilitation facility which does not employ a phlebotomist. As used herein, a phlebotomist is a person who is trained to take blood from a patient by venipuncture, and is not a physician.

A protein array is a set of regions or spots on a surface, with each region or spot containing protein having one or more known characteristics, such as composition or origin of the protein(s). Also included is a protein array, where the surface is a set of beads, with each bead being a different region or spot.

As used herein, ELISA includes all assays for an antibody of interest in blood or serum that uses an antigen bound (directly or indirectly) to a surface, and a secondary antibody which binds to the antibody of interest, and a reporter molecule bound to the secondary antibody. Preferably, the reporter molecule is fluorescent. Multiple ELISA includes an assay which simultaneously carries out a plurality of ELISA for different antibodies originating from blood or serum of a patient.

The original volume of a blood sample is the volume of the blood when taken from the patient. A blood sample may later be dried, for example onto a blood sample collection card.

### BRIEF DESCRIPTION OF THE DRAWINGS

FIG. 1 illustrates a kit for collecting a blood sample for allergy testing.

FIG. 2 illustrates a surface element having protein arrays.

FIG. 3 illustrates a protein array.

### DETAILED DESCRIPTION

In order to improve patient compliance with allergy testing, it would be desirable if such a test could be carried out by physicians who are not allergist, such as a primary care physician. This would avoid the need for further appointments with an allergist, or to refer patients to a laboratory employing a phlebotomist, which result in additional costs and requires follow-through by the patients. By using a protein array, an amount of blood need in order to determine the presence of specific IgE in a blood sample may be reduced to 0.5 ml, or less. This amount of blood is equivalent to about 10 drops of blood, or less, and may be obtained using a lancet, by cutting a finger or arm. Such a small amount of blood does not require venipuncture, and may be obtain by a physician or nurse; a phlebotomist is not required.

Another separate advantage of the small amount of blood in the blood sample is that it may be easily dried, for example by placing the blood sample onto a blood collection card and allowing it to air dry for 15 minutes or less. No special equipment is needed to dry the blood. After a blood sample is dried most chemical reaction cease, allowing accurate analysis of IgE in the blood sample by ELISA 10 days or more after collection of the blood sample, without refrigeration. A small amount of dried blood is also much less of a biohazard than vials of liquid blood, allowing transport of the dried blood sample to a testing center on a blood collection card (secured in a mailing envelope) by regular first class mail. Not only is the cost of transporting the blood sample to the testing center dramatically reduced, but blood samples may be collected in rural and isolated locations. In addition, analysis of the blood samples may be centralized into national or regional testing centers, further reducing costs.

A problem with existing blood tests for allergies is the mismatch between the antigens present in the allergen source, and the proteins used in a multiple ELISA for binding the IgE in a patient's blood sample to a surface. The proteins used in the multiple ELISA are single pure proteins, usually prepared recombinantly. Such single pure proteins do not contain many of the antigens present in the allergen source. Furthermore, recombinantly prepared proteins are usually made using genetically transformed prokaryotic organism, while the allergen source is (or is from) a eukaryotic organism, so post-translational modifications of the proteins may not be the same, resulting in proteins which many not contain all the same epitopes present in the allergen source. Accordingly, it is possible for the multiple ELISA to mistakenly determine that a patient is not allergic to a particular allergen source, because the proteins used in the multiple ELISA are not reactive with an antibody present in the patient's blood sample which is, in fact, reactive with an antigen present in the allergen source (that is, the test indicates a false negative). Such a false negative may lead to ineffective allergy immunotherapy.

In addition to a false negative, it is also possible that a false weak response is observed in the multiple ELISA, when in fact the patent would have a server allergic reaction to the antigen source, due to the mismatch. When allergy immunotherapy is administered to the patient using an amount of allergen source extract appropriate for a weak response, the patient may experience a life-threatening anaphylactic reaction or asthma exacerbation.

To avoid these problems, rather than using purified, isolated and/or recombinantly produced proteins in the multiple ELISA, a mixture of proteins from the allergen source, such as allergen source extracts, are used for each individual ELISA. Preferably, the allergen source extracts are used, and preferably a protein array is used, with one allergen source extract used for each spot or region on the protein array. By having multiple proteins from each allergen source for each individual ELISA in the multiple ELISA, in proportions that more closely match the proportions present in each allergen source, quantification of total IgE binding in each individual ELISA (for example, at each region or spot of the protein array) will more accurately reflect the patient's true response to the allergen source. Furthermore, when such a multiple ELISA is used for an allergy blood test, followed by immunotherapy using the same standardized allergen source extracts as were used to prepare the protein array of the multiple ELISA, there will be an excellent match of antigens used for testing and immunotherapy. This matching of antigens used for testing and immunotherapy was previously only available when a skin test was used for allergy testing, and is believed to provide the best patient outcomes.

Optionally, combining both the use of a small blood sample obtain without the use of a phlebotomist for testing using a protein array, and where the protein array contains multiple proteins from the allergen source at each region or spot, will combine advantages noted above. Optionally, combining both the use of a dried blood sample for testing using a protein array, and where the protein array contains multiple proteins from the allergen source at each region or spot, will combine all the advantages noted above.

The present invention includes a method of testing a patient for allergies, including obtaining from the patient a blood sample, and then testing the blood sample for IgE reactive with antigen from multiple allergen sources. Preferably, the blood is taken from the patient using a lancet, for example from a finger or an arm, so that a phlebotomist is not needed, and venipucture is avoided. The blood may be taken by a physician, such as a primary care physician, an allergist, a gastroenterologist, a pulmonologist, a hematologist, an ear-nose-and-throat specialist (ENT), or may be taken by a nurse, a chiropractor, a pharmacist, a physical therapist or a physician's assistant. Preferably, the blood is taken in a clinic, so that there is no need to refer the patient to a laboratory.

The amount of blood is preferably at most 0.50 ml (about 10 drops), including at most 0.40 ml (about 8 drops), at most 0.30 ml (about 6 drops), at most 0.25 ml (about 5 drops), at most 0.20 ml (about 4 drops), at most 0.10 ml (about 2 drops), and at most 0.05 ml (about 1 drop), including 0.05 to 0.50 ml, 0.10 to 0.40 ml, and 0.15 to 0.35 ml. The blood sample may be a fluid, or may be dried (for example, fluid or dried serum, or fluid or dried whole blood). Optionally, an anticoagulant and/or preservative, or other additives, may be added to the blood sample. Preferably, the blood is applied to a blood collection card (for example, 903 Sample Collection Card (GE Healthcare, Little Chalfont, Buckinghamshire, United Kingdom)) and allowed to dry; drying of such small blood samples, such as by air drying, requires no special equipment and typically requires 15 minutes or less. The blood is preferably whole blood, but on some blood collection cards the blood can be separated and only the serum used as the blood sample. The blood sample may then be sent to an allergy testing center; for example, when the blood sample is a dried blood sample, it may be sent to an allergy testing center by placing it in a sealed mailing envelope or other container, and delivered by currier or U.S. Postal Service. Optionally, when the blood sample is a dried blood sample, no refrigeration is required during transport, the risks associated with blood biohazard are reduced, and the blood sample is stable enough that analysis of the blood sample may be carried out as long as 30 days after the sample was collected. Preferably, the blood sample is analyzed 2 to 30 days after collection, or 3 to 10 days after collection, including 4, 5, 6, 7, 10, 15, 20 and 25 days after collection. The use of a dried blood sample greatly simplifies the logistics of transporting the dried blood sample to a testing center, and allows collection of the blood sample in rural and isolated locations which do not have the logistics service and associated testing centers needed for liquid blood sample transport and analysis.

A kit for collecting a blood sample for allergy testing could be used by the person obtaining the blood sample, including a blood collection card, a lancet, and a shipping envelope. Optionally one or more of the following may also be in the kit: a patient reference card, an additional lancet, an additional blood collection card, instructions, a container for holding the contents of the kit together, a cleansing pad for cleaning the skin where the lancet will pierce, gauze for applying pressure to skin to stop bleeding, and a bandage to apply to the wound created by the lancet. Optionally, the shipping envelope may be pre-address for an allergy testing center. Optionally, the patient reference card may include a removable self-adhesive patient identifier label, which may be applied to the blood collection card, and an identical non-removable patient identifier. Especially in the case of using a kit for collecting a blood sample for allergy testing, the patient could carry out the blood collection from himself or herself using such a kit.

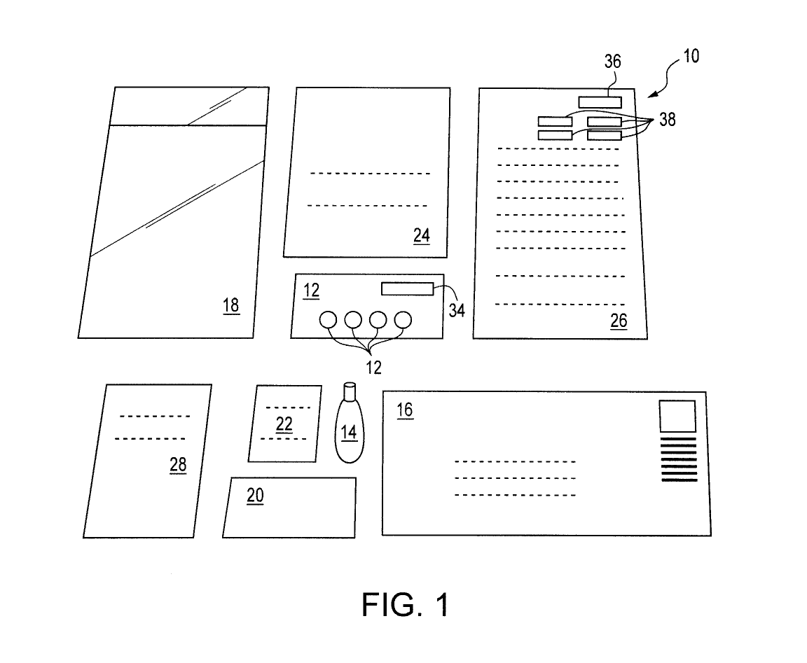

FIG. 1 illustrates such a kit with the parts separated for clarity. The kit, 10, includes a blood collection card, 12, a lancet, 14, a shipping envelope, 16, a container, 18, a bandage, 20, a cleansing pad, 22, optional instructions, 24, a patient reference card, 26, and gauze, 28. The blood collection card includes one or more blood sample collection areas, 30, (for example 1, 2, 3 or 4) which indicate where the patient's blood should be applied, and optionally a patient reference area, 34, which indicates who the patient is, for example by a patient reference code. The patient reference card (also referred to as a laboratory requisition form) may provide information about the patient, such as who is the patient, using a patient reference code, 36, and may also have one or more removable self-adhesive patient identifier labels, 38, which may be applied to the patient reference area of the blood collection card.

The instruction provides the steps for blood collection. These include using the lancet to lance a finger or arm, optionally squeezing the lanced finger or arm to generate drops of blood, and applying the blood drops to the blood sample collection area or areas on the blood collection card. After allowing the blood to dry, typically 5 to 10 minutes, the card is placed into the shipping envelope and sent to a testing center. Optionally, the patient reference card may also be included, having patient identifying information, and corresponding patient identifying information may be placed onto the blood collection card (for example, a self-adhesive patient identifier label removed from the patient reference card, such as a bar code). Optionally, the skin which will be lanced may be cleansed prior to lancing. Optionally, additional blood may be applied to different blood sample collection areas on the blood collection card.

In order to test the patient blood sample, the blood sample collection area may be separated from the blood collection card by cutting it out, for example with a hole punch. The blood sample collection area is then soaked in water or a saline solution to extract the blood sample. Serum is then prepared from the blood sample, by filtration or centrifugation, and tested using multiple ELISA.

Preferably, the patient is a mammal, most preferably a human. Other mammals may also be the patient, for example horses, dogs or cats.

The blood sample may be tested for IgE reactive with antigens from allergen sources. The testing is preferably carried out by multiple ELISA, preferably using a protein array, for example a protein array having a glass or plastic surface on which the proteins are attached. Preferably, each region or spot of the array contains a mixture of different proteins from a single allergen source. Examples of allergen sources are shown in Table 1; all those listed are available as powders, liquids, freeze-dried, and/or extracts from GREER® Allergy Immunotherapy (Lenoir, N.C.). Table 2 lists a preferred set of allergen sources for inclusion on a protein array. In a variation, one or more of the regions or spots may contain a mixture of different proteins from a single class of allergen sources, such as mites, dander, epithelia, feathers, grasses, weeds, trees, shrubs, plants, flowers, fungi, smuts, venoms, plant foods, fish, shellfish, animal foods, poultry products, dairy products, nuts, insects, dusts, and inhalants. Optionally, none of the allergen sources used to prepare the protein array are foods, or all of the allergen sources used to prepare the protein array are foods. Preferably, regions or spots containing a mixture of proteins from a single allergen source may be present as duplicates, triplicates, or in greater multiplicity to improve the statistical significance of the multiple ELISA. Optionally, the selection of allergen sources represented on a protein array may be selected for prevalence of the selected allergen source within a specific geographical region. Optionally, the selection of allergen sources represented on a protein array may be limited to a particular class of allergen sources. Optionally, additional regions or spot may be present on the protein array, for example one or more control regions or spots which specifically bind to IgE, for example, a control region containing mouse antibodies that specifically bind human IgE. Furthermore, multiple protein arrays may be present on each surface element, for example 1 to 100, 2 to 50, or 5 to 25, protein arrays may be present on a single slide or stick, such as 21, 22, 23, 24, 25, 26, 27, 28 or 29 protein arrays present on a glass or plastic slide. Also possible is a protein array where the surface element is a set of beads, with each bead having one region or spot.

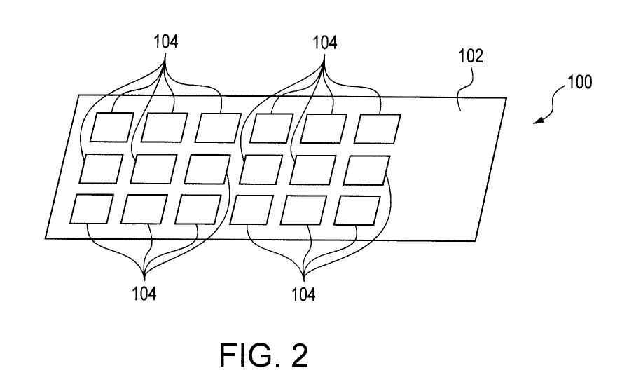

FIG. 2 illustrates a surface element having protein arrays. Surface element, 100, has a surface, 102, on which are multiple protein arrays, 104. In this illustration, the surface element has 8 protein arrays.

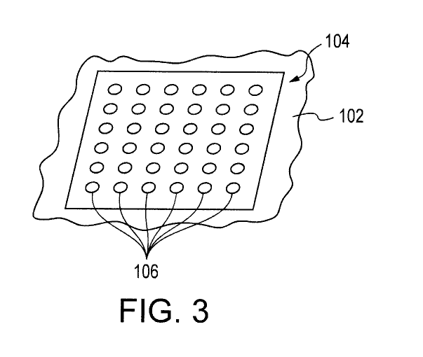

FIG. 3 illustrates a protein array. The protein array, 104, is on a surface, 102, of a surface element (not shown). The protein array has multiple regions or spots, 106. Preferably, most of the regions or spots contain a mixture of proteins, preferably in duplicate or triplicate, and the array contains one or more control regions or spots which may be a single protein or another substance.

Preferably, the number of regions or spots in each protein array is at least 30, at least 40, at least 50, at least 60, at least 70, at least 80, at least 90, at least 100, at least 110, at least 150, at least 200, at least 250, or at least 300, including 30, 40, 50, 60, 63, 66, 69, 70, 72, 75, 78, 80, 81, 84, 87, 90, 93, 96, 99, 100, 102, 105, 108, 111, 114, 117, and 120, as well as all ranges of values between any combinations of these values, such as 10 to 300, 20 to 200, 30 to 120, or 40 to 111. Preferably, the number of allergen sources used in each protein array is at least 10, at least 20, at least 30, at least 40, at least 50, at least 60, at least 70, at least 80, at least 90, or at least 100, including 10, 20, 21, 22, 23, 24, 25, 26, 27, 28, 29, 30, 31, 32, 33, 34, 35, 36, 37, 38, 39, 40, 50, 60, 70, 80, 90, and 100, as well as all ranges of values between any combinations of these values, such as 10 to 100, 20 to 90, 25 to 80, or 30 to 70.

Methods of applying proteins to surfaces to form arrays are well known. The immobilization may be covalent or noncovalent, via a linker moiety, or tethering to an immobilized moiety. These methods are well known in the field of solid phase synthesis and micro-arrays, for example as described in Beier et al., Nucleic Acids Res. 27:1970-1-977 (1999), and U.S. Pat. No. 6,101,946 to Martinsky. Examples include binding moieties for attaching proteinaceous molecules to a solid support include streptavidin or avidin/biotin linkages, carbamate linkages, ester linkages, amide, thiolester, (N)-functionalized thiourea, functionalized maleimide, amino, disulfide, amide, hydrazone linkages, and among others. Furthermore, a number of companies offer commercial services for preparing protein arrays, for example Arrayit Corporation (Sunnyvale, Calif.). Preferably, the source of the proteins is an extract of the allergen source, such as those available from GREER® Allergy Immunotherapy (Lenoir, N.C.). Preferably, the extract of the allergen source is standardized.

After the multiple ELISA is carried out, the results of the assay are provided to a physician and/or the patient, or a physician's assistant, a nurse, a chiropractor, a pharmacist, or a physical therapist. Preferably, the physician is an allergist or a primary care physician, or a gastroenterologist, a pulmonologist, a hematologist, or an ear-nose-and-throat specialist (ENT). If a primary care physician took the blood sample from the patient, then preferably the results of the assay are provided to that primary care physician. The results of the assay include a listing of allergen sources, specifically those allergen sources corresponding to the antigens to which the IgE of the patient's blood bonded. The results may also include an amount or relative amount of binding for each mixture of allergen source proteins. Preferably, the listing will indicate if the amount of IgE binding is clinically significant, or will only list the allergen source if the binding to the proteins of the allergen source was clinically significant.

After the results are provided, the patient may be treated with allergy immunotherapy. Preferably, the patient is treated by administering to the patient a small amount of the allergen source, for example an allergen source extract, preferably a standardized allergen source extract. If a protein array was used in the multiple ELISA that was prepared from one or more standardized allergen source extracts, then preferably during allergy immunotherapy the same standardized allergen source extracts are administer to the patient. In this way, the patient's response to the allergy immunotherapy will closely match the response expected based on the results of the multiple ELISA. Furthermore, the effectiveness of the allergy immunotherapy will be superior and an unexpected life-threatening anaphylactic reaction, or asthma attack, will be less likely to occure.

Immunotherapy may be carried out by administering the small amount of the allergen source subcutaneously, or preferably sublingually; ingestion of the allergen source, optionally associated with or enveloped in a protective particle such as chitosan, is also possible. Preferably, the administration is carried out by a primary care physician, a physician's assistant, a nurse, a chiropractor, a pharmacist, or a physical therapist. Alternatively, the administration is carried out by a physician who is not an allergist, for example a primary care physician, a gastroenterologist, a pulmonologist, a hematologist, or an ear-nose-and-throat specialist (ENT).

TABLE 1
Allergen Sources

Mites
Mite, House Dust (Glycyphagus
domesticus)
Mite, House Dust (Blomia tropicalis)
Mite, House Dust (Euroglyphus maynei)
Mite, Food/Storage (Lepidoglyphus
destructor)
Mite, Food/Storage (Acarus siro)
Mite, Food/Storage (Tyrophagus
putrescentiae)
Mite, House Dust (Dermatophagoides
farinae)
Mite, House Dust (Dermatophagoides
pteronyssinus)
Danders
Cat Dander (Felis catus (domesticus))
Dog Dander (Canis familiaris)
Horse Dander (Equus caballus)
Human Dander (Homo sapiens)
Epithelia
Cat Epithelia (Felis catus (domesticus))
Cattle Epithelia (Bos Taurus)
Dog Epithelia, Mixed-Breed (Canis
familiaris)
Goat Epithelia (Capra hircus)
Guinea Pig Epithelia (Cavia porcellus)
Hog Epithelia (Sus scrofa)
Horse Epithelia (Equus caballus)
Mouse Epithelia (Mus musculus)
Rabbit Epithelia (Oryctolagus cuniculus)
Rat Epithelia (Rattus norvegicus)
Sheep Epithelia (Ovis aries)
Hamster Epithelia (Mesocricetus auratus)
Gerbil Epithelia (Meriones unguiculatus)
Feathers
Canary Feathers (Serinus canaria)
Chicken Feathers (Gallus gallus)
Duck Feathers (Anas platyrhynchos)
Goose Feathers (Anser domesticus)
Parakeet Feathers (Psittacidae)
Pigeon Feathers (Columba spp.)
Chicken Feathers (Gallus gallus)
Goose Feathers (Anser anser)
Duck Feathers (Anas platyrhynchos)
Grasses
Bermuda (Cynodon dactylon)
Brome Grass, Smooth (Bromus inermis)
Canarygrass, Reed (Phalaris arundinacea)
Couch/Quack Grass (Elymus repens)
Meadow Fescue (Festuca elatior
(pratensis))
Johnson Grass (Sorghum halepense)
Kentucky Blue/June (Poa pratensis)
Orchard (Dactylis glomerata)
Oats, Common/Cultivated (Avena sativa)
Redtop (Agrostis gigantea (alba))
Rye, Cultivated (Secale cereale)
Ryegrass, Perennial (Lolium perenne)
Ryegrass, Italian (Lolium perenne ssp.
Multiflorum)
Sweet Vernal (Anthoxanthum odoratum)
Timothy (Phleum pretense)
Velvetgrass (Holcus lanatus)
Wheat, Cultivated (Triticum aestivum)
Corn, Cultivated (Zea mays)
Wheatgrass, Western (Pascopyrum
smithii)
Bahia (Paspalum notatum)
Ryegrass, Giant Wild (Leymus
condensatus)
Weeds
Careless Weed, Amaranth/Green
(Amaranthus hybridus)
Cocklebur (Xanthium strumarium)
Dock, Yellow/Curly (Rumex crispus)
Nettle (Urtica dioica)
Goldenrod (Solidago spp.)
Hemp, Water (Amaranthus rudis)
Firebush/Kochia (Kochia scoparia)
Lamb's Quarter (Chenopodium album)
Marsh Elder, Burweed/Giant Poverty (Iva
xanthifolia)
Marsh Elder, True/Rough (Iva annua)
Mugwort, Common (Artemisia vulgaris)
Mugwort, Darkleaved/Sagebrush, Prairie
(Artemisia ludoviciana)
Palmer's Amaranth (Amaranthus palmeri)
Ragweed, Slender (Ambrosia confertiflora)
Pigweed, Rough/Redroot (Amaranthus
retroflexus)
Pigweed, Spiny (Amaranthus spinosus)
Plantain, English (Plantago lanceolata)
Ragweed, Giant (Ambrosia trifida)
Ragweed, Short (Ambrosia artemisiifolia)
Ragweed, Southern (Ambrosia bidentata)
Ragweed, Western (Ambrosia
psilostachya)
Russian Thistle (Salsola kali)
Sagebrush, Common (Artemisia tridentate)
Sorrel, Sheep/Red (Rumex acetosella)
Dog Fennel (Eupatorium capillifolium)
Ragweed, False (Ambrosia acanthicarpa)
Sagebrush, Pasture (Artemisia frigida)
Saltbush, Annual (Atriplex wrightii)
Marsh Elder, Narrowleaf (Iva angustifolia)
Allscale (Atriplex polycarpa)
Wingscale (Atriplex canescens)
Iodine Bush (Allenrolfea occidentalis)
Burrobrush (Hymenoclea salsola)
Rabbit Bush (Ambrosia deltoidea)
Quailbrush (Atriplex lentiformis)
Baccharis (Baccharis spp.)
Ragweed, Desert (Ambrosia dumosa)
Baccharis (Baccharis sarothroides)
Baccharis (Baccharis halimifolia)
Cultivated Plants and Flowers
Sugar Beet (Beta vulgaris)
Alfalfa (Medicago sativa)
Clover, Red (Trifolium pretense)
Daisy, Ox-Eye (Leucanthemum vulgare)
Dandelion (Taraxacum officinale)
Sunflower (Helianthus annuus)
Mustard (Brassica spp.)
Castor Bean (Ricinus communis)
Fungi
Alternaria alternate
Aspergillus flavus
Aspergillus fumigatus
Aspergillus amstelodami
Aspergillus niger
Acremonium strictum
Tricothecium roseum
Chaetomium globosum
Cladosporium herbarum
Epidermophyton floccosum
Fusarium moniliforme
Bipolaris sorokiniana
Cladosporium sphaerospermum
Microsporum canis
Candida albicans
Neurospora intermedia
Penicillium camembertii
Penicillim notatum
Penicillium chrysogenum
Aureobasidium pullulans
Rhizopus oryzae
Rhizopus stolonifer
Trichoderma harzianum
Trichophyton mentagrophytes
Mucor plumbeus
Penicillium digitatum
Epicoccum nigrum
Botrytis cinerea
Stemphylium solani
Paecilomyces variotii
Aspergillus nidulans
Phoma betae
Helminthosporium solani
Gliocladium viride
Penicillium roquefortii
Nigrospora oryzae
Mycogone perniciosa
Drechslera spicifera
Fusarium solani
Rhodotorula mucilaginosa
Trichophyton rubrum
Mucor circinelloides f. circinelloides
Aspergillus terreus
Geotrichum candidum
Stemphylium botryosum
Saccharomyces cerevisiae
Mucor circinelloides f. lusitanicus
Setosphaeria rostrata
Phoma herbarum
Penicillium expansum
Penicillium brevicompactum
Serpula lacrymans
Malassezia pachydermatis
Aspergillus versicolor
Poultry Products
Chicken Meat (Gallus gallus)
Duck Meat (Anas spp.)
Egg Whole, Chicken (Gallus gallus)
Egg White, Chicken (Gallus gallus)
Egg Yolk, Chicken (Gallus gallus)
Goose Meat (Anser anser)
Turkey Meat (Meleagris gallopavo)
Dairy Products
Casein, Bovine (Bos taurus)
Milk, Bovine (Bos taurus)
Milk, Goat (Capra hircus)
Nuts
Almond (Prunus dulcis)
Brazil Nut (Bertholletia excelsa)
Cashew Nut (Anacardium occidentale)
Coconut (Cocos nucifera)
Filbert/Hazelnut (Corylus americana)
Peanut (Arachis hypogaea)
Pecan (Carya illinoinensis)
Pistachio (Pistacia vera)
Walnut, Black (Juglans nigra)
Walnut, English (Juglans regia)
Dusts
Barley Grain Dust
Corn Grain Dust
House Mixture Dust
Oat Grain Dust
Wheat Grain Dust
Upholstery Dust
Mattress Dust
Insects
House Fly (Musca domestica)
Moth (Lepidoptera)
Mayfly (Ephemeroptera)
Ant, Fire (Solenopsis richteri)
Flea (Ctenocephalides canis/C. felis)
Cockroach, American (Periplaneta
americana)
Caddisfly (Trichoptera)
Ant, Black/Carpenter (Camponotus
pennsylvanicus)
Deer Fly (Chrysops sp.)
Horse Fly (Tabanus spp.)
Cockroach, German (Blattella germanica)
Ant, Fire (Solenopsis invicta)
Mosquito (Culicidae)
Culicoides (Culicoides spp.)
Trees and Shrubs
Alder, Hazel (Alnus serrulata)
Ash, Red/Green (Fraxinus pennsylvanica)
Ash, Oregon (Fraxinus latifolia)
Ash, White (Fraxinus Americana)
Aspen (Populus tremuloides)
Beech, American (Fagus grandifolia)
Birch, River (Betula nigra)
Birch, White (Betula populifolia)
Birch, Black/Sweet (Betula lenta)
Box Elder (Acer negundo)
Cedar, Mountain (Juniperus ashei)
Cedar, Red (Juniperus virginiana)
Cottonwood, Eastern (Populus deltoids)
Cottonwood, Western (Populus deltoides
ssp. Monilifera)
Elm, American (Ulmus Americana)
Elm, Siberian (Ulmus pumila)
Hazelnut, European (Corylus avellana)
Hackberry (Celtis occidentalis)
Hazelnut, American (Corylus Americana)
Hickory, Pignut (Carya glabra)
Hickory, Shellbark (Carya laciniosa)
Hickory, Shagbark (Carya ovata)
Hickory, White (Carya alba)
Maple, Red (Acer rubrum)
Maple, Soft/Silver (Acer saccharinum)
Maple, Sugar/Hard (Acer saccharum)
Mesquite (Prosopis velutina)
Mulberry, Red (Morus rubra)
Mulberry, Paper (Broussonetia papyrifera)
Oak, Black (Quercus velutina)
Oak, Bur (Quercus macrocarpa)
Oak, Virginia Live (Quercus virginiana)
Oak, Post (Quercus stellata)
Oak, Red (Quercus rubra)
Oak, White (Quercus alba)
Olive (Olea europaea)
Pecan (Carya illinoinensis)
Pine, Longleaf (Pinus palustris)
Pine, Eastern White (Pinus strobus)
Pine, Yellow (Pinus echinata)
Pine, Loblolly (Pinus taeda)
Poplar, White (Populus alba)
Privet, Common (Ligustrum vulgare)
Sweet Gum (Liquidambar styraciflua)
Sycamore, American/Eastern (Platanus
occidentalis)
Walnut, Black (Juglans nigra)
Willow, Black (Salix nigra)
Locust Blossom, Black (Robinia
pseudoacacia)
Pine, Virginia Scrub (Pinus virginiana)
Acacia (Acacia spp.)
Bayberry/Wax Myrtle (Morella cerifera)
Poplar, Lombardy (Populus nigra)
Elm, Cedar (Ulmus crassifolia)
Cypress, Bald (Taxodium distichum)
Mulberry, White (Morus alba)
Oak, California Live (Quercus agrifolia)
Eucalyptus (Eucalyptus globulus)
Oak, Water (Quercus nigra)
Ash, Arizona (Fraxinus velutina)
Beefwood/Australian Pine (Casuarina
equisetifolia)
Walnut, English (Juglans regia)
Palm, Queen (Arecastrum
romanzoffianum)
Alder, Red (Alnus rubra)
Melaleuca (Melaleuca quinquenervia)
Walnut, California Black (Juglans
californica)
Sycamore, Western (Platanus racemosa)
Juniper, Utah (Juniperus osteosperma)
Cypress, Italian (Cupressus sempervirens)
Cedar, Salt/Tamarisk (Tamarix gallica)
Juniper, Oneseed (Juniperus
monosperma)
Cypress, Arizona (Cupressus arizonica)
Orange Pollen (Citrus sinensis)
Juniper, Western (Juniperus occidentalis)
Cottonwood, Fremont (Populus fremontii)
Oak, Arizona/Gambel (Quercus gambelii)
Mango Blossom (Mangifera indica)
Pepper Tree (Schinus spp.)
Juniper, Pinchot (Juniperus pinchotii)
Juniper, Rocky Mountain (Juniperus
scopulorum)
Oak, California Black (Quercus kelloggii)
Oak, Western White (Quercus garryana)
Cedar, Japanese (Cryptomeria japonica)
Pepper Tree (Schinus molle)
Pepper Tree/Florida Holly (Schinus
terebinthifolius)
Birch, Spring (Betula occidentalis)
Alder, White (Alnus rhombifolia)
Cottonwood, Black (Populus balsamifera
ssp. Trichocarpa)
Oak, California White (Quercus lobata)
Sycamore/London Plane (Platanus
hybrida)
Pine, Ponderosa (Pinus ponderosa)
Olive, Russian (Elaeagnus angustifolia)
Pine, White/Western (Pinus monticola)
Willow, Arroyo (Salix lasiolepis)
Alder, European (Alnus glutinosa)
Sycamore, Oriental (Platanus orientalis)
Oak, English (Quercus robur)
Birch, European White (Betula pendula)
Oak, Holly (Quercus ilex)
Elm, Siberian (Ulmus pumila)
Smuts
Loose Barley Smut (Ustilago nuda)
Bermuda Grass Smut (Ustilago
cynodontis)
Corn Smut (Ustilago maydis)
Johnson Grass Smut (Sporisorium
cruentum)
Oat Smut (Ustilago avenae)
Loose Wheat Smut (Ustilago tritici)
Venoms
Yellow Hornet (Dolichovespula arenaria)
White (Bald)-Faced Hornet
(Dolichovespula maculate)
Honey Bee (Apis mellifera)
Mixed Yellow Jacket (Vespula spp.)
Mixed Hornet (Dolichovespula spp.)
Mixed Paper Wasp (Polistes spp.)
Plant Foods
Apple (Malus pumila)
Apricot (Prunus armeniaca)
Banana (Musa paradisiaca var.
paradisiacal)
Barley (Hordeum vulgare)
Bean, Lima (Phaseolus lunatus)
Bean, Pinto (Phaseolus sp.)
Bean, Red Kidney (Phaseolus sp.)
Bean, String/Green (Phaseolus vulgaris)
Blackberry (Rubus allegheniensis)
Blueberry (Vaccinium myrtilloides)
Broccoli (Brassica oleracea var. botrytis)
Buckwheat (Fagopyrum esculentum)
Cabbage (Brassica oleracea var. capitata)
Cantaloupe (Cucumis melo)
Carrot (Daucus carota)
Cauliflower (Brassica oleracea var.
botrytis)
Celery (Apium graveolens var. dulce)
Cherry (Prunus sp.)
Chocolate/Cacao Bean (Theobroma
cacao)
Cinnamon (Cinnamomum verum)
Coffee (Coffea arabica)
Corn (Zea mays)
Cranberry (Vaccinium macrocarpon)
Cucumber (Cucumis sativus)
Garlic (Allium sativum)
Ginger (Zingiber officinale)
Grape (Vitis sp.)
Grapefruit (Citrus x paradise)
Hops (Humulus lupulus)
Lemon (Citrus limon)
Lettuce (Lactuca sativa)
Malt (Hordeum vulgare)
Mushroom (Agaricus bisporus)
Nutmeg (Myristica fragrans)
Oat (Avena sativa)
Olive, Green (Olea europaea)
Onion (Allium cepa var. cepa)
Orange (Citrus sinensis)
Pea, Black-eyed (Vigna unguiculata)
Pea, Green (English) (Pisum sativum)
Peach (Prunus persica)
Peanut (Arachis hypogaea)
Pear (Pyrus communis)
Pepper, Black (Piper nigrum)
Pepper, Green (Capsicum annuum var.
annuum)
Pineapple (Ananas comosus)
Potato, Sweet (Ipomoea batatas)
Potato, White (Solanum tuberosum)
Raspberry (Rubus idaeus var. idaeus)
Rice (Oryza sativa)
Rye (Secale cereale)
Sesame Seed (Sesamum orientale
(indicum))
Soybean (Glycine max)
Spinach (Spinacia oleracea)
Squash, Yellow Summer (Cucurbita pepo
var. melopepo)
Strawberry (Fragaria chiloensis)
Tomato (Solanum lycopersicum var.
lycopersicum)
Turnip (Brassica rapa var. rapa)
Vanilla Bean (Vanilla planifolia)
Watermelon (Citrullus lanatus var. lanatus)
Wheat, Whole (Triticum aestivum)
Bean, Navy (Phaseolus vulgaris)
Mustard (Brassica spp.)
Fish and Shellfish
Bass, Black (Centropristis striata)
Catfish (lctalurus spp.)
Clam (Mercenaria spp.)
Codfish (Gadus spp.)
Crab (Callinectes spp.)
Flounder (Platichthys sp.)
Halibut (Hippoglossus hippoglossus)
Sardine (Clupeiformes)
Lobster (Homarus americanus)
Mackerel (Scomberomoros cavalla)
Oyster (Ostrea/Crassostrea)
Perch (Serranus scriba)
Salmon (Salmo salar)
Scallop (Placopecten magellanicus)
Shrimp, White/Brown/Pink (Litopaenaeus
setiferus, Farfantepenaeus aztecus,
Farfantepenaeus dourarum)
Trout, Lake (Oncorhynchus mykiss)
Tuna Fish (Thunnus spp.)
Animal Foods
Beef (Bos taurus)
Lamb (Ovis aries)
Pork (Sus scrofa)
Inhalants
Cottonseed (Gossypium hirsutum)
Flaxseed (Linum usitatissimum)
Kapok Seed (Ceiba pentandra)
Orris Root (Iris germanica var. florentina)
Pyrethrum (Chrysanthemum
cinerariifolium)
Silk (Bombyx mors)
Tobacco Leaf (Nicotiana tabacum)
Gum, Karaya (Sterculia urens)
Gum, Acacia/Arabic (Acacia senegal)
Gum Tragacanth (Astragalus gummifer)
Cotton Linters (Gossypium hirsutum)
Anisakis* (Anisakis simplex)

TABLE 2
Preferred allergen sources.
ALLERGEN SOURCE

Cat dander
Dog dander
Alder
Bahia grass
Bermuda grass
Johnson grass
Perrenial Rye Grass
Timothy grass
Cockroach
Blomia tropicalis
House dust mite (D. farinae)
House dust mite (D. pteronyssinus)
Alternaria alternata
Aspergillus fumigatus
Cladosporium herbarum
Penicillium notatum
Acacia
Australian pine
Birch
Cottonwood
Elm
Maple
Maple leaf sycamore
(London Plane)
Mountain cedar
Mulberry
Oak
Olive
Hickory
Walnut
White ash
Short ragweed
Mugwort
Nettle
Rough marsh elder
Rough pigweed
Russian thistle
Sheep sorrel

Aspects of the inventions described herein have been described on the website of ARRAYIT® Corporation (arrayit.com) and links thereon. All such disclosures were made by another who obtained the subject matter disclosed directly or indirectly from the inventor of the present application.

1. A method of testing a patient for allergies, comprising:
obtaining from the patient a blood sample having an original volume of at most 0.5 ml, and
testing the blood sample for IgE reactive with antigens from a plurality of allergen sources by multiple ELISA,
wherein (a) the blood sample was taken from the patient in a clinic, and/or (b) the blood sample was taken by a physician without venipuncture.

2. (canceled)

3. The method of claim 1, wherein the blood sample was taken by a primary care physician without venipuncture.

4. (canceled)

5. The method of claim 1, wherein the blood sample has an original volume of at most 0.25 ml.

6-8. (canceled)

9. The method of claim 1, wherein the multiple ELISA is carried out with a protein array comprising a plurality of regions or spots on a surface, each region or spot comprising a mixture of different proteins from a single allergen source.

10. The method of claim 9, wherein the mixture of different proteins from the single allergen source comprises proteins present in a standardized extract of the single allergen source.

11. A protein array for testing blood for IgE, comprising:
a plurality of regions or spots on a surface, each region or spot comprising a mixture of different proteins from a single allergen source.

12. The protein array of claim 11, wherein the mixture of different proteins from the single allergen source comprises proteins present in an extract of the single allergen source.

13. The protein array of claim 11, wherein the mixture of different proteins from the single allergen source consist of proteins present in an extract of the single allergen source.

14. The protein array of claim 12, wherein the extract of the single allergen source is a standardized extract.

15-20. (canceled)

21. A method of treating a patient having allergies with allergy immunotherapy, comprising:
testing the patient for allergies by the method of claim 9, to identify at least one allergen source to which the patient is allergic, and
administering to the patient the at least one allergen source.

22. The method of claim 21, wherein the at least one allergen source administered to the patient is a standardized extract of each at least one allergen source.

23. (canceled)

24. A method of treating a patient having allergies with allergy immunotherapy, comprising:
testing the patient for allergies by the method of claim 10, to identify at least one allergen source to which the patient is allergic, and
administering to the patient the at least one allergen source,
wherein the at least one allergen source administered to the patient is the standardized extract of the allergen source.

25-27. (canceled)

28. A method of treating a patient having allergies with allergy immunotherapy, comprising:
testing a blood sample from the patient for IgE reactive with antigens from a plurality of allergen sources by multiple ELISA, to identify at least one allergen source to which the patient is allergic, and
administering to the patient the at least one allergen source,
wherein the multiple ELISA is carried out with a protein array comprising a plurality of regions or spots on a surface, each region or spot comprising a mixture of different proteins from a single allergen source.

29. The method of claim 28, wherein the mixture of different proteins from a single allergen source comprises proteins present in a standardized extract of the allergen source.

30-35. (canceled)

36. The method of claim 1, further comprising drying the blood sample after the obtaining of the blood sample from the patient.

37-43. (canceled)

44. A method of testing a patient for allergies, comprising:
obtaining from the patient a blood sample having an original volume of at most 0.5 ml,
drying the blood sample, and
testing the blood sample for IgE reactive with antigens from a plurality of allergen sources by multiple ELISA.

45-49. (canceled)

50. The method of claim 44, wherein the multiple ELISA is carried out with a protein array comprising a plurality of regions or spots on a surface, each region or spot comprising a mixture of different proteins from a single allergen source.

51. The method of claim 50, wherein the mixture of different proteins from a the single allergen source comprises proteins present in a standardized extract of the single allergen source.

52. A method of treating a patient having allergies with allergy immunotherapy, comprising:
testing the patient for allergies by the method of claim 50, to identify at least one allergen source to which the patient is allergic, and
administering to the patient the at least one allergen source.

53. A method of treating a patient having allergies with allergy immunotherapy, comprising:
testing the patient for allergies by the method of claim 51, to identify at least one allergen source to which the patient is allergic, and
administering to the patient the at least one allergen source,
wherein the at least one allergen source administered to the patient is a the standardized allergen source extract of the allergen source which has the same composition as that present in the protein array.

54. (canceled)
